(19) United States
(12) Patent Application Publication
(10) Pub. No.: US 2018/0282250 A1
(43) Pub. Date: Oct. 4, 2018

(54) VACUUM DISTILLATION FOR ENRICHING CANNABIDIOL

(71) Applicant: Bionorica Ethics GmbH, Neumarkt (DE)

(72) Inventors: Andreas RUTZ, Dornhausen (DE); Michael ENGLERT, Nümberg (DE)

(21) Appl. No.: 15/765,025

(22) PCT Filed: Sep. 30, 2016
(86) PCT No.: PCT/EP2016/073527

§ 371 Date: Mar. 30, 2018

(30) Foreign Application Priority Data
Sep. 30, 2015 (EP) 15187795.8

(51) Int. Cl. C07C 37/74; B01D 3/12

(52) U.S. Cl. CPC C07C 37/74; A61K 36/185; B01D 3/12

(57) ABSTRACT

The present invention relates to a method for producing, obtaining and enriching cannabidiol (CBD) and to the use thereof. The invention also relates to a cannabidiol extract and the use of an obtained cannabidiol extract in the pharmaceutical and cosmetic fields.

The present invention relates to a method for producing, obtaining and enriching cannabidiol (CBD) and to the use thereof. The invention also relates to a cannabidiol extract and the use of an obtained cannabidiol extract in the pharmaceutical and cosmetic fields.

Cannabis (hemp) belongs together with the genus Humulus (hop) to the family of Cannabaceae, wherein however Humulus does not contain cannabinoids. Within the genus Cannabis there is a botanical and chemotaxonomic distinction made, more specifically in the Cannabis species Cannabis sativa Linnaeus, Cannabis indica LAM, and Cannabis ruderalis or in the “species complex” Cannabis sativa L., consisting of the Cannabis sativa sub-groups ssp. sativa and ssp. indica. In addition, cannabis can be distinguished into a drug hemp and fibre hemp, wherein the distinction is made on the basis of the ratio of the primary cannabinoids cannabidiol (CBD) and Δ⁹-tetrahydrocannabinol (Δ⁹-THC) (INN: Dronabinol). Fibre hemp (also: industrial hemp) is used primarily: for industrial fibre production and may have at most a Δ⁹-THC content of 0.2% (for example in Germany, inter alia), whereas the drug type can have a Δ⁹-THC content of approximately 5-15% (marijuana, hashish). Cannabis sativa L. contains more than 400 different ingredients, including more than 60 compounds from the class of cannabinoids. The most important cannabinoids are listed hereinafter:

Cannabigerol types (CBG): cannabigerol ((E)-CBG-C₅), cannabigerol monomethylether ((E)-CBGM-C₅ A), cannabinerolic acid A ((Z)-CBGA-C₅ A), cannabigerovarin ((E)-CBGV-C₃), cannabigerolic acid A ((E)-CBGA-C₅ A), cannabigerolic acid A monomethylether ((E)-CBGAM-C₅ A), cannabigerovarinic acid A ((E)-CBGVA-C₃ A);

Cannabichromene types (CBC): cannabichromene (CEO-C₅), cannabichromenic acid A (CBCA-C₅ A), cannabichromevarin (CBCV-C₃), cannabichromevarinic acid A (CBCVA-C3 A);

Cannabidiol types (CBD): cannabidiol (CEO-C₅), cannabidiol monomethylether (CBDM-C₅), cannabidiol-C4 (CBD-C₄), cannabidivarin (CBDV-C₃), cannabidiorcol (CEO-C₁), cannabidiolic acid (CBDA-C₅), cannabidivarinic acid (CBDVA-C₃);

Cannabinodiol types (CBND): cannabinodiol (CBND-C₅), cannabinodivarin (CBND-C₃);

Tetrahydrocannabinol types (THC): Δ9-tetrahydrocannabinol (Δ9-THC-C₅), Δ9-tetrahydrocannabinol-C4 (Δ9-THC-C₄), Δ9-tetrahydrocannabivarin (Δ9-THCV-C₃), Δ9-tetrahydrocannabiorcol (Δ9-THCO-C₁), Δ9-tetrahydrocannabinolic acid (Δ9-THCA-C₅ A), Δ9-tetrahydrocannabinolic acid B (Δ9-THCA-C₅ B), Δ9-tetrahydrocannabinolic acid-C4 (Δ9-THCA-C₄ A and/or B), Δ9-tetrahydrocannabivarinic acid A (Δ9-THCVA-C₃ A), Δ9-tetrahydrocannabiorcolic acid (Δ9-THCOA-C₁ A and/or B), (−)-Δ8-trans-(6aR,10aR)-Δ8-tetrahydrocannabinol (Δ8-THC-C₅), (−)-Δ8-trans-(6aR,10aR)-tetrahydrocannabinolic acid A (Δ8-THCA-C₅ A); (−)-(6aS,10aR)-Δ9-tetrahydrocannabinol ((−)-cis-Δ9-THC-C₅);

Cannabinol types (CBN): cannabinol CBN-C₅, cannabinol-C4 (CBN-C₄), cannabivarin (CBN-C₃), cannabinol-C2 (CBN-C₂), cannabiorcol (CBN-C₁), cannabinolic acid A (CBNA-C₅ A), cannabinolmethylether (CBNM-C₅)

Cannabitriol types (CBT): (−)-(9R,10R)-trans-cannabitriol ((−)-trans-CBT-C₅), (+)-(9S,10S)-cannabitriol ((+)-trans-CBT-05), (±)-(9R,10S/9S,10R)-cannabitriol ((±)-cis-CBT-C₅), (−)-(9R,10R)-trans[10-O-ethyl-cannabitriol] ((−)-trans-CBT-OEt-C₅), (±)-(9R,10R/9S,10S)-cannabitriol-C3 ((±)-trans-CBT-C₃), 8,9-dihydroxy-Δ6a(10a) tetrahydrocannabinol (8,9-Di-OH-CBT-C₅), cannabidiolic acid A (CBDA-C₅ 9-OH-CBT-C5 ester), (−)-(6aR,9S,10S,10aR)-9,10-dihydroxy-hexahydrocannabinol, cannabiripsol cannabiripsol-C5, (−)-6a,7,10a-trihydroxy-Δ9-tetrahydrocannabinol ((−)-cannabitetrol), 10-Oxo-Δ6a(10a) tetrahydrocannabinol (OTHC);

Cannabielsoin types (CBE): (5aS,6S,9R,9aR)-C₅-cannabielsoin (CBE-C₅), (5aS,6S,9R,9aR)-C₃-cannabielsoin (CBE-C₃), (5aS,6S,9R,9aR)-cannabielsoic acid A (CBEA-C₅ A), (5aS,6S,9R,9aR)-cannabielsoic acid B (CBEA-C₅ B), (5aS,6S,9R,9aR)-C3-cannabielsoic acid B (CBEA-C₃ B), cannabiglendol-C3 (OH-iso-HHCV-C₃), dehydrocannabifuran (DCBF-C₅), cannabifuran (CBF-C₅);

Isocannabinoids: (−)-Δ7-trans-(1R,3R,6R)-isotetrahydrocannabinol, (±)-Δ7-1,2-cis-(1R,3R,6S/1S,3S,6R)-isotetrahydrocannabivarin, (−)-Δ7-trans-(1R,3R,6R)-isotetrahydrocannabivarin;

Cannabicyclol types (CBL): (±)-(1aS,3aR,8bR,8cR)-cannabicyclol (CBL-C₅), (±)-(1aS,3aR,8bR,8cR)-cannabicyclolic acid A (CBLA-C₅ A), (±)-(1aS,3aR,8bR,8cR)-cannabicyclovarin (CBLV-C₃);

Cannabicitran types (CBT): cannabicitran (CBT-C₅);

Cannabichramanon types (CBCN): carmabichromanon (CBCN-C₅), Cannabichromanon-C3 (CBCN-C₃) cannabcoumaronon (CBCON-C₅)

Besides the above-mentioned cannabinoids, their associated carboxylic acids are also found in the crude drug. These carboxylic acids are biosynthetic precursors.

Cannabis preparations exert a large number of therapeutic effects, including antispastic, analgesic, antiemetic, neuroprotective and anti-inflammatory effects, and effects in psychiatric illnesses (Grotenhermen F, Müller-Vahl K: The therapeutic potential of cannabis and cannabinoids. Dtsch Arztebl Int 2012; 109(29-30): 495-501. DOI: 10.3238/arztebl.2012.0495).

In Germany, a cannabis extract containing THC (Dronabinol) and CBD in a ratio of 1:1 (Nabiximols) has been approved for use as a pharmaceutical product since 2011 for the treatment of moderately severe to severe, therapy-resistant spasticity in multiple sclerosis (MS) in the form of a sublingual spray (Sativex).

Cannabidiol (CBD, CBD-C₅) is the most important non-psychotropic cannabinoid of the Cannabis genus, and CBD is not a cannabinoid receptor agonist.

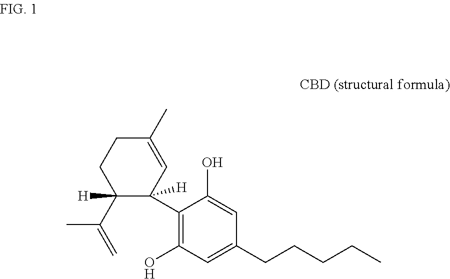 [SMILES: C=C(C)[C@@H]1CCC(C)=C[C@H]1c1c(O)cc(CCCCC)cc1O]

CBD can be produced synthetically (Michoulam R, Shvo Y., Hashish. I. The structure of cannabidiol, Tetrahedron. 1963, 19(12), 2073).

Furthermore, methods for extraction of cannabidiol are described in the prior art:

DE 100 51 427 C1 (Müller) describes a CBD-containing primary extract by means of CO₂ extraction from cannabis fibre hemp with use of supercritical or subcritical pressure and temperature conditions. To this end, an SFC or SFE system is used (“superfluid-chromatography”).

However, the prior art dues not describe a suitable vacuum distillation for extraction of CBD in high purity from a cannabis raw material. The prior art discloses vacuum distillation of THC (WO 00/25127A1), and EP1051084B1 describes steam distillation from hemp, for example.

The object is therefore to provide an improved distillation for extraction of CBD so that an extract that is free of THC to the greatest possible extent, and even a completely THC-free extract containing CBD is provided.

The object is achieved by the stated claims.

The invention therefore relates to a method for extracting cannabidiol from cannabis plant material, wherein a vacuum distillation is performed (hereinafter: method according to the invention).

In the sense of the invention, distillation means a thermal separation process in order to obtain vaporisable liquids from a gas phase and to separate substances that are difficult to vaporise, wherein a cannabidiol-containing extract is enriched in the distillate. In the sense of this invention, “vacuum distillation” means that the distillation is performed in a vacuum at 0.001 to 50 mbar, preferably 0.001 to 10 mbar, particularly preferably 0.001 to 1 mbar in what is known as the fine vacuum range. It is also preferred that the temperature is 120 to 240 degrees Celsius, in particular 150 to 230 degrees Celsius.

The method according to the invention for vacuum distillation is preferably performed by means of short-path distillation. “Short-path distillation” in the sense of this invention means that the gas phase in the applied fine vacuum only has to travel over a very short path between the receiver and the condenser. For example, a short-path evaporator can be used which corresponds structurally to a conventional thin-film evaporator, wherein, however, the condenser is integrated in the interior of the evaporator cylinder, such that the path that must be traveled by the vapours to the condenser is very short and pressures of 0.001 mbar can be reached.

In accordance with the invention a method according to the invention by means of a thin-film evaporator or falling film evaporator is also included. Thin-film evaporators and falling film evaporators are thermal separation apparatuses that are known per se.

A suitable thin-film evaporator in the sense of the invention comprises a substantially cylindrical inner wall, which is heated by steam and to which a thin film of a primary extract is applied by means of rotating distributing bodies. The motor-driven distributing bodies are required in order to apply and distribute the mixture, which evaporates quickly on the plates.

A suitable falling film evaporator in the sense of this invention comprises a substantially vertically running, externally heated pipe, on the inner side of which a primary extract trickles down in a thin film and in so doing evaporates. The un-evaporated components are removed at the bottom end of the pipe as sump, and the evaporated components leave the other end of the pipe as head product. The falling film evaporator consequently does without any moving parts. A common design feature of thin-film evaporators and falling film evaporators is at least one heated, planar evaporation body, to which a thin film of the liquid primary extract is applied and evaporated in part (ULLMANN: Billet, Reinhard: Evaporation, Ullmann's Encyclopedia of industrial Chemistry, Published Online: 15 Jun. 2000 DOI: 10.1002/14356007.b03_03, VTA Verfahrenstechnische Anlagen GmbH & Co. KG, Niederwinkling (DE)).

However, a short-path evaporator or a thin-film evaporator is preferred in accordance with the invention.

It is also preferred that the method according to the invention for vacuum distillation is performed using at least one column or rectifying column. A suitable column according to the invention is, for example, a DN 60 column (050 mm, L=360 cm) with vacuum jacket and TI connection piece made of boron silicate. The column can have conventional bottoms and packings or compartments, such that at least 10 separation stages preferably are achieved. Such rectifying columns according to the invention are obtainable for example from VTA Verfahrenstechnische Anlagen GmbH & Co. KG, Niederwinkling (DE). In a further embodiment, the length of the column is at least 2.50 m, preferably 2.70 m, or more than 3 m.

In a further preferred embodiment of the invention the vacuum distillation can be coupled with column distillation. In particular, a short-path evaporator, thin-film evaporator, or falling film evaporator can be equipped accordingly with a column. In the sense of this invention, column distillation is likewise a suitable rectification under vacuum, also under reflux, which preferably has 10 separation stages. The rectification allows the reliable separation of THC and enrichment of CBD to more than 80%.

It is also preferred that the vacuum distillation according to the invention is performed with coupled column distillation at a pressure of from 0.001 to 50 mbar, preferably 0.001 to 10 mbar, in particular 0.001 to 1 mbar. It is also preferred that the temperature is 120 to 240 degrees Celsius, in particular 150 to 230 degrees Celsius. In a preferred embodiment the pressure is up to 5-10 mbar and the temperature is 200 to 230 degrees Celsius. This allows reliable CBD enrichment alongside procurement of a THC-free extract, wherein THC is completely depleted.

It is also preferred that the vacuum distillation is performed on a first primary extract with at least 15% by weight of CBD. In a first step the cannabis plant material is therefore cut and comminuted, and subjected to a first extraction, for example CO₂ extraction, as described in DE 100 51 427 C1. Alternatively, extraction with hexane or column chromatography can also be performed, and in this way a primary extract preferably with at least 15% by weight of CBD can be obtained, which is used for further vacuum distillation (supra). However, CO₂ extraction is preferred. Preferred cannabis plant material is fibre hemp or industrial hemp, in particular of the following kinds: Fedora 17, Felina 34, Ferimon 12, Futura 75, inter alia with relatively high content of CBD in % by weight.

The invention therefore also relates to a cannabidiol-containing extract obtainable by a method according to the invention. The extract is honey-coloured, spreadable, has a pleasant smell, and has a CBD content of more than 35% by weight, more than 50% by weight, in particular more than 70% by weight in dry weight and is practically THC-free or even completely THC-free.

In a further embodiment, an extract having the following composition could be obtained in accordance with the method according to the invention:

| | | |
|---|---|---|
| Cannabidiol (CBD) | % | 73.0 |
| Delta9-THC | % | 0.58 |
| C3-cannabidiol | % | 0.09 |
| C4-cannabidiol | % | 0.17 |
| CBN | % | 0.19 |

The method according to the invention therefore allows an enrichment of cannabidiol of more than 73%, in particular 75%, and 80% or more with advantageous enrichment of other cannabinoids, in particular THC.

The method according to the invention therefore makes it possible to set at least the CBD to THC ratio to 125:1 and greater in the obtained extract. The method according to the invention therefore makes it possible to set at least the CBD to CBN ratio to 384:1 and greater in the obtained extract.

The invention therefore also relates to a cannabidiol-containing extract obtainable by a method according to the invention and having a CBD:THC ratio of at least 125:1 and/or a CBD:CBN ratio of at least 384:1.

Furthermore, the low-boiling proportion based on CBD [%] is 6.16 and the high-boiling proportion based on CBD [%] is 4.87. In other words, almost all substances that boil before and after cannabidiol advantageously can be selectively separated by the method according to the invention.

The invention therefore also relates to a cannabidiol-containing extract obtainable by a method according to the invention having a high-boiling proportion of less than 5% based on CBD and a low-boiling proportion of less than 7% based on CBD.

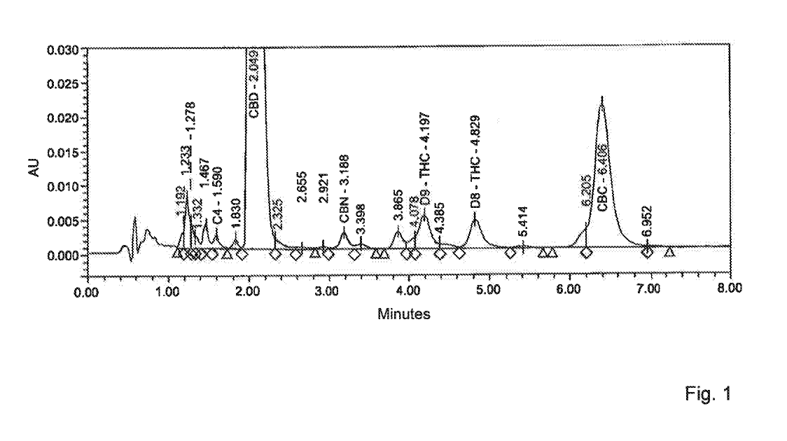

FIG. 1 shows an HPLC chromatogram of an extract of this kind obtained from column distillation under vacuum.

Furthermore, an exemplary starting composition was used as follows for the method according the invention, with a starting ratio of approximately 45:1:

| | | |
|---|---|---|
| Cannabidiol (CBD) | % | 23.8 |
| Delta9-THC | % | 0.53 |
| C3-cannabidiol | % | 0.71 |
| C4-cannabidiol | % | 0.13 |
| CBN | % | 0.71 |

Furthermore, the low-boiling proportion based on CBD [%] is 19.8, and the high-boiling proportion based on CBD [%] is 26.0.

The method according to the invention particularly advantageously allows a significant reduction of the low- and high-boiling proportions compared to a used starting material. This leads to an extract having specific properties, such as the advantageous capability, of residue-free vaporisation which is particularly suitable for inhalation of CBI) in a medical application, in particular for a pharmaceutical product.

This enriched cannabidiol-containing extract, on account of its excellent consistency, can be used immediately particularly advantageously in formulations such as ointments, creams, gels, lotions (body milk), pastes or preferably emulsions.

The invention therefore also relates to a pharmaceutical product, dietary supplement or cosmetic containing a cannabidiol-containing extract obtainable by a method according to the invention. The pharmaceutical product is particularly suitable for antispastic, analgesic, antiemetic, neuroprotective, anti-inflammatory effect and in psychiatric illnesses.

A pharmaceutical composition or agent according to the invention, in particular a pharmaceutical product (medicament), can be galenically prepared in the usual manner. Suitable solid or liquid galenic preparation forms are, for example, granules, powder, lozenges, tablets, (micro)capsules, hard capsules, suppositories, syrups, juices, pensions, or emulsions, in the production of which conventional auxiliaries such as carriers, disintegrants, binders, coating agents, swelling agents, lubricants, flavourings, sweeteners and solubilisers are used. Potential auxiliary substances are magnesium stearate, sodium chloride, magnesium carbonate, titanium dioxide, lactose, mannitol and other sugars, talc, milk protein, gelatins, starches, celluloses and derivatives thereof, animal and vegetable oils, such as cod liver oil, sunflower oil, groundnut oil or sesame oil, polyethylene glycols, and solvents, such as sterile water and mono- or polyvalent alcohols, for example glycerol

Further detergents and surfactants can also be provided as auxiliaries and additives, as will be mentioned hereinafter by way of example for a cosmetic composition.

Emulsions, in particular for a cosmetic composition, are understood generally to mean heterogeneous systems consisting of two liquids that are not miscible with one another or that are only miscible with one another to a limited extent and which are usually referred to as phases. In an emulsion, one of the two liquids is dispersed in the form of very fine droplets within the other liquid. If the two liquids are water and oil and oil droplets are finely distributed in water, this is an oil-in-water emulsion (O/W emulsion). The basic character of an O/W emulsion is defined by the water. In the case of a water-in-oil emulsion. (W/O emulsion), the principle is reversed and the basic character is determined here by the oil. Mixed systems, such as water-in-oil-in-water emulsions (W/O/W emotions) and oil-in-water-in-oil emulsions (O/W/O emotions) are also known. All specified emulsions are suitable in accordance with the invention.

The anhydrous systems suitable in accordance with the invention include pure oil preparations, such as skin oils. Pastes containing the preparation according to the invention can also be used and are characterised in that they consist of the same or similar constituents as an emulsion, but are substantially anhydrous. Within the scope of the present invention, the terms oil phase and lipid phase are used synonymously. In a further preferred embodiment the preparation according to the invention can contain an emulsifier as a further constituent. In a very preferred embodiment this emulsifier can be an O/W emulsifier.

Emulsifiers can be selected advantageously from the group of non-ionic, anionic, cationic or amphoteric emulsifiers.

Various emulsifiers from the groups of partial fatty acid esters, fatty alcohols, sterols, polyethylene glycols such as ethoxylated fatty acids, ethoxylated fatty alcohols and ethoxylated sorbitan esters, sugar emulsifiers, polyglycerol emulsifiers or silicone emulsifiers can be used as non-ionic emulsifier.

Various emulsifiers from the groups of soaps, for example, sodium stearate, fatty alcohol sulphate, mono-, di- and trialkyl phosphoric esters and ethoxylates thereof, fatty acid lactate esters, fatty acid citrate esters, or fatty acid citroglycerol esters can be used as anionic emulsifiers.

For example, quaternary ammonium compounds with a long-chain aliphatic group, for example distearyldimonium chlorides, can be used as cationic emulsifiers.

Various emulsifiers from the groups alkylamininoalkane carboxylic acids, betaines, sulfobetaines or imidazoline derivatives can be used as amphoteric emulsifiers.

Naturally occurring emulsifiers, for example including beeswax, lanolin wax, lecithin and sterols, amongst others, which can likewise be used in the production of a preparation according to the invention are preferred in accordance with the invention. In a preferred formulation of the preparation according to the invention, O/W emulsifiers can be selected from the group of plant protein hydrolysates and derivatives thereof.

In the sense of the present invention, substances selected from the group of esters of saturated and/or unsaturated, branched and/or unbranched alkane carboxylic acids and/or alkene carboxylic acids with a chain length of 3-30 carbon atoms and saturated and/or unsaturated, branched and/or unbranched alcohols with a chain length of 3-30 carbon atoms and from the group of esters of aromatic carboxylic acids and saturated and/or unsaturated, branched and/or unbranched alcohols with a chain length of 3 to 30 carbon atoms can also be contained advantageously as additives. Esterols of this kind can then be selected advantageously from the group of isopropylmyristate, isopropylpalmitate, isopropylstearate, isopropyloleate, n-butylstearate, n-hexyllaurate, n-decyloleate, isooctylstearate, isononylstearate, isononyliso-nonanoate, 2-ethylhexylpalmitate, 2-ethylhexyllaurat, 2-hexyldecylstearate, 2-octyldodecyl-palmitate, oleyloleate, oleylerucate, erucyloleate, erucylerucate and synthetic, semisynthetic and natural mixtures of such esters, such as jojoba oil.

The oil phase can also be selected advantageously from the group of branched and unbranched hydrocarbons and waxes, dialkyl ethers, the group of saturated or unsaturated, branched or unbranched alcohols, and fatty acid triglycerides, specifically triglycerol esters of saturated and/or unsaturated, branched and/or unbranched alkane carboxylic acids with a chain length of 8-24 carbon atoms, in particular 12-18 carbon atoms. The fatty acid triglycerides can be selected advantageously for example from the group of synthetic, semisynthetic and natural oils.

In particular, antioxidants and/or radical catchers can be added additionally as auxiliary or additive to the preparations according to the invention, Antioxidants of this kind are advantageously selected from the group of lipophilic systems, for example: natural and synthetic tocopherols, nordihydroguaiaretic acid, coniferyl benzoate, butylhydroxyanisole, butylhydroxytoluene, gallic acid ester, and various antioxidant plant extracts. From the hydrophilic systems, inorganic sulphur compounds, sodium hydrogen sulphite, cysteine, or ascorbic acid can be used particularly advantageously.

The cosmetic preparations according to the invention can also contain cosmetic auxiliaries, as are used conventionally in preparations of this kind, for example preservatives, bactericides, perfumes, substances for preventing foaming, dyes, pigments which have a colouring effect, thickeners, surface-active substances, softening, dampening and/or moistening substances, or other conventional constituents of a cosmetic or dermatological formulation, such as alcohols, polyols, polymers, foam stabilisers, electrolytes, organic solvents or silicone derivatives.

In a further particular embodiment, the preparation according to the invention is composed of substantially naturally occurring ingredients, as mentioned above.

1.-15. (canceled)

16. A method for extracting cannabidiol from cannabis plant material, which comprises a vacuum distillation by means of a rectifying column is performed,
wherein the vacuum distillation is performed on a primary extract with at least 15% by weight cannabidiol,
wherein the pressure is 0.001 to 50 mbar and the temperature is 120 to 240° C.,
and an enrichment of cannabidiol of more than 70% by weight in the dry weight of an extract is achieved, with depletion of THC.

17. The method for extracting cannabidiol from cannabis plant material according to claim 16, wherein the vacuum distillation is a short-path distillation.

18. The method for extracting cannabidiol from cannabis plant material according to claim 16, wherein the vacuum distillation is performed by means of a thin-film evaporator, short-path evaporator or falling film evaporator.

19. The method for extracting cannabidiol from cannabis plant material according to claim 18, wherein the length of the rectifying column is at least 2.50 m.

20. The method for extracting cannabidiol from cannabis plant material according to claim 16, wherein the pressure is 0.001 to 10 mbar and the temperature is 150 to 240 degrees Celsius.

21. The method for extracting cannabidiol from cannabis plant material according to claim 16, wherein the vacuum distillation is coupled with a column distillation.

22. A cannabidiol-containing extract obtained by the method according to claim 16, having a CBD:THC ratio of at least 125:1.

23. The cannabidiol-containing extract according to claim 22, having a CBD:CBN ratio of at least 384:1.

24. The cannabidiol-containing extract according to claim 21, wherein the extract has the capability of residue-free vaporisation.

25. A pharmaceutical product containing a cannabidiol-containing extract for use in inhalation obtained by the method according to claim 16.

26. A cosmetic or dietary supplement which comprises the cannabidiol-containing extract according to claim 22.
